(19) United States
(12) Patent Application Publication
(10) Pub. No.: US 2007/0129702 A1
(43) Pub. Date: Jun. 7, 2007

(54) Reusable and disposable diaper for dogs

(76) Inventor: Linda Sue Gribben, California, MD (US)

Correspondence Address: CONNOLLY BOVE LODGE & HUTZ, LLP, P O BOX 2207, WILMINGTON, DE, 19899, US

(21) Appl. No.: 11/546,495

(22) Filed: Oct. 11, 2006

Related U.S. Application Data
(60) Provisional application No. 60/725,441, filed on Oct. 11, 2005.

(51) Int. Cl. A61F 13/15

(52) U.S. Cl. 604/392; 604/385.09

(57) ABSTRACT

A reusable and disposable dog diaper for removable emplacement and securement circumferentially about a dog's rear torso and inguinal region. By combining a waterproof (or water-resistant) lower body sheet, a highly absorbent material and a fastener that can be attached, detached and reattached many times without damaging the fastener or the diaper, the diaper of the present invention achieves the desirable goals of being both reusable and disposable. The diaper is easy to mount on the dog and easy to remove.

### CROSS-REFERENCE TO RELATED APPLICATION

This application claims the benefit of the filing date of U.S. provisional application No. 60/725,441 filed on Oct. 11, 2005.

### BACKGROUND OF THE INVENTION

1. Technical Field

This invention relates to a diaper for dogs that is both disposable and reusable. In use, the diaper is fastened circumferentially about the rear torso of a dog and its inguinal region to absorb and retain any body fluids, such as urine, discharged by the dog.

2. Background

There is a need for a reusable and disposable dog diaper that can be easily fastened circumferentially about the dog's rear torso and its inguinal region to absorb and retain body fluids, such as urine, discharged by the dog to prevent soiling while traveling, to prevent soiling of carpets and furniture, and to dissipate the effects which would otherwise be present of odors and unsanitary conditions from such discharges.

### SUMMARY OF THE INVENTION

The diaper of the present invention is designed to be both reusable and disposable. Specifically, the diaper is designed so that it can be: (1) fastened circumferentially about the rear torso and inguinal region of a male dog to absorb and retain body fluids discharged by the dog; (2) removed from the dog before being fully used (i.e., before the diaper is fully saturated with liquid); (3) refastened to the dog one or more times until the diaper is either fully used (i.e., fully saturated with liquid) or until the diaper has outlived its usefulness (due to odor or any other undesirable property exhibited by the partially used diaper); and then (4) disposed of by placing the diaper in the trash.

### BRIEF DESCRIPTION OF THE DRAWINGS

These objects and other objects of the invention should be discerned and appreciated from the description of the preferred embodiment taken in conjunction with the drawings, wherein like reference numerals refer to similar parts throughout the several drawing figures, in which:

FIG. 1 is a plan view of the interior of the diaper.

FIG. 2 is a plan view of the exterior of the diaper.

FIG. 3 is a view showing a disposable and reusable dog diaper according to this invention mounted on (or fastened to) a dog.

### DESCRIPTION OF THE PREFERRED EMBODIMENT(S)

The diaper of the present invention has a substantial advantage over the disposable dog diapers of the prior art because it can be reused many times until it is either fully used (fully saturated with liquid) or until it has outlived its usefulness. The reusable aspect of the present diaper is achieved by using a fastener that can be attached, unattached and then reattached several times without damaging the diaper. In a preferred embodiment, the fastener for the diaper is made of a hook and loop system such as Velcro®. Although Velcro® is the preferred fastener for the dog diaper of the present invention, other types of fasteners can be used as long as they form an attachment that is strong enough to hold the diaper in place, as the dog moves about, and are reusable in that the fastener can be attached, detached and reattached several times without losing its ability to form a strong attachment or otherwise damaging the diaper (e.g., by ripping the diaper).

Another advantage of the diaper of the present invention is that it can be easily and quickly mounted on, or fastened to, the dog because it does not require any of the dog's legs or tail to be placed through the diaper. The diaper is simply wrapped around the rear torso of the dog (usually by passing one end of the diaper between the dog's front and back legs), the ends of the diaper are pulled past each other until the diaper is snug on the dog and the ends are then connected or attached to one another by the fastener (for example by connecting the hook portion of the Velcro® to the loop portion).

In the disposable dog diapers of the prior art, the fastener was usually an adhesive system that brought a sticky surface into contact with another portion of the diaper. Once the sticky surface was attached to the other portion of the diaper, it was not possible to detach the sticky portion from the other portion of the diaper without damaging the diaper, usually by pulling out a piece of the surface of the diaper along with the sticky portion or by ripping the diaper. Thus, these diapers were single-use diapers in that once the diaper was mounted on, or fastened to the dog, it was designed to stay fastened to the dog until it was time to dispose of the diaper. If the diaper was removed from the dog for any reason before it was fully used, the diaper had to be thrown away because it could not be reattached without a new piece of adhesive material (not part of the original diaper) or if reattachment was possible, the strength of the attachment was greatly diminished and the reattached diaper was not very secure.

In contrast, the fastener of the dog diaper of the present invention is designed so that it can be used many times without any significant loss in the strength of its attachment. That is, even after several attachment, detachment and reattachment cycles, the diaper is still secure on the dog while in use. In a preferred embodiment of the present invention, the fastener can be used for at least three (3) attachment, detachment and reattachment cycles (i.e., the diaper can be used at least three (3) times). In a highly preferred embodiment, the fastener can be used for at least five (5) attachment, detachment and reattachment cycles (i.e., the diaper can be used at least five (5) times).

Although the diaper of the present invention can be used with any size dog (by adjusting the size of the diaper or the length of the fastener), it is particularly useful and effective for small dogs (such as Chihuahuas, Maltise, Pomeranians, Yorkshire Terriers, Japanese Chins, Chinese Cresteds, Miniature Pinchers, Dachshunds, Italian Greyhounds, Jack Russell Terriers, Poodles, and small breed mixes) and puppies. In a preferred embodiment of the present invention, the diaper is used with male dogs that weigh about 25 pounds or less.

The term “disposable” as used herein means that the diapers are designed to be discarded (placed into the trash) after the absorbent material has been partially or completely saturated with liquid. This term is meant to distinguish the diapers of the present invention from those diapers that are designed to be washed or cleaned between uses.

The term “reusable” as used herein means that the diapers can be temporarily removed from the dog and then reused, by reattaching the diaper to the dog, if the absorbent material has not been fully saturated with liquid. This term does not mean that the diapers can be washed or cleaned and then reused. The liquid that is captured by the absorbent material stays in the absorbent material and once the absorbent material is fully saturated with liquid, the diaper is no longer reusable and must be disposed of (i.e., as trash).

The term “inguinal region” as used herein describes the groin area of a dog. On a male dog, this region specifically includes the penis and does not include the tail or anus. In use, on a male dog, the diapers of the present invention cover the penis (but not the tail or anus) and the absorbent material is disposed, with respect to the dog's penis, so that any fluid discharge from the dog's penis will land on (or otherwise contact) the absorbent material.

The absorbent material of the present invention is a permanent part of the diaper and is not a replaceable insert. In a preferred embodiment of the present invention, the absorbent material is permanently attached to the waterproof or water-resistant body sheet, for example with an adhesive agent or stitching or by bonding the body sheet to the absorbent material (e.g., by applying heat).

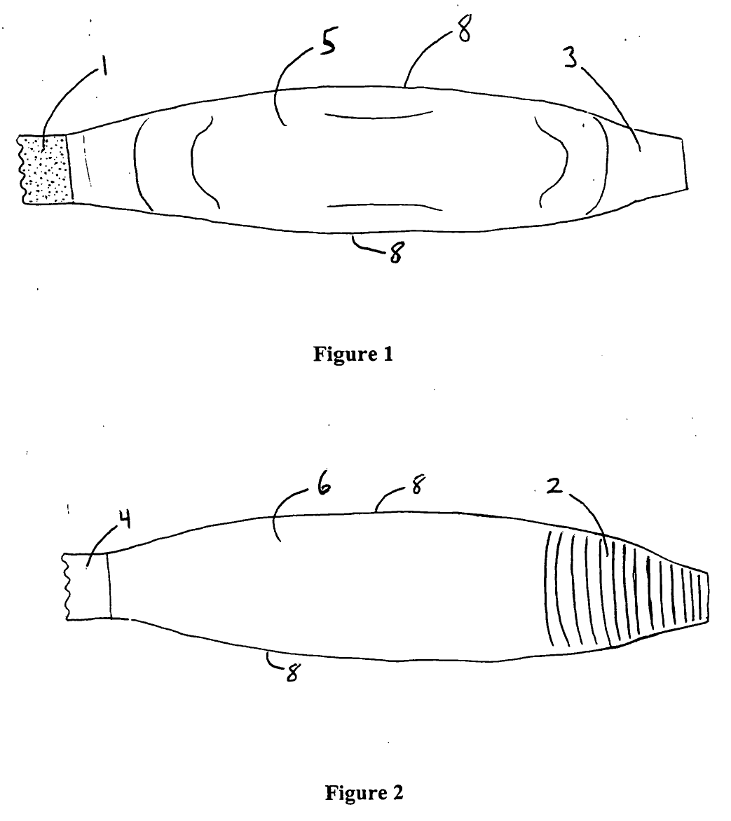

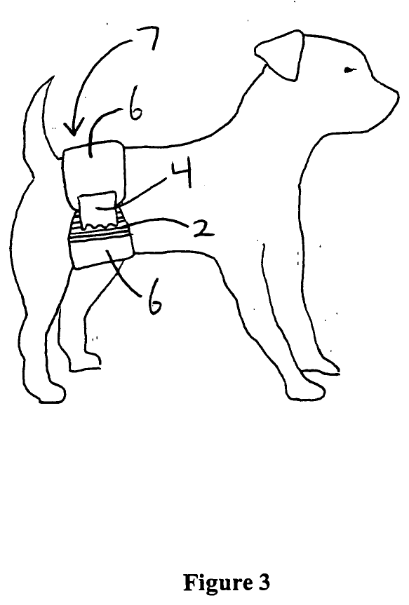

In FIG. 3 of the drawings, reference numeral 7 generally refers to the disposable dog diaper of the present invention, shown mounted on the rear torso of a male dog and covering his inguinal region. As shown in FIG. 2, the diaper comprises an exterior lower body sheet 6 that is flexible and is made of suitable waterproof material. Body sheet 6 carries in superposed relationship suitable, highly absorbent material 5 (FIG. 1). Extending from one end of body sheet 6 is one end of a fastener, shown as 2 in FIG. 2. This end of the fastener attaches to the other end of the diaper, shown as 1 in FIG. 1. One or both of the sides 8 of the diaper can be gathered to allow comfortable and complemental emplacement of the body sheet 6 against the dog's lower rear torso with the highly absorbent material 5 interfacing with the dog's lower rear torso and inguinal region. Upon total and complete emplacement and engagement of the diaper against the dog's rear torso, the diaper wraps around the dog's upper rear torso to effect therewith comfortable emplacement and engagement without the diaper slipping, gathering or otherwise “bunching up” in consequence from the dog's running movements or other movements. As shown in FIGS. 1 and 2, the two end surfaces of the diaper 1 and 2 attach or connect to one another to form an attachment which holds the diaper in place on the dog and by pulling the ends 1 and 2 of the diaper past each other (i.e., increasing the amount of overlap) before attaching end surface 1 to end surface 2, it is possible to decrease the effective size of the diaper, thus allowing the diaper to snugly fit a smaller dog. The highly absorbent material 5 absorbs and retains body fluids, such as urine, discharged by the dog and the body sheet 6 prevents any leakage of such absorbed fluids. After the diaper has been completely used or has otherwise outlived its usefulness, it can be easily and simply removed and disposed of. The diaper is easy to attach because it requires only minimal cooperation from the dog. As discussed earlier, the diaper is attached around the dog's torso, not around or through the dog's legs, as, for example, is the case with a human infant's diaper and many of the dog diapers of the prior art. As a result, the dog's natural range of movements are not restricted. This permits the dog to stand, even on its hind legs, sit, lie down, walk and even run naturally. These functional attributes of the diaper contribute to the dog's comfort and thereby reduce the likelihood of the dog's attempts to remove or destroy the diaper, either of which would reduce the effectiveness and/or usefulness of the diaper.

The dog diaper of the present invention is designed to be reusable. Although, in practice, the diaper may be used only once (e.g., when the dog urinates in the diaper and mostly or completely saturates the absorbent material with liquid while the diaper is on its first use), the diaper is designed so that it can be used at least twice (i.e., the diaper would be attached to the dog, detached from the dog, reattached to the dog and then detached from the dog a second time before the diaper is disposed of). Although we have described a use “cycle” earlier in this application as being an attachment, detachment and reattachment of the diaper to a dog, the “reattachment” step of the cycle is really the first step in the second use of the diaper. Thus, to be completely clear, a single use cycle is an attachment of the diaper and a detachment of the diaper (i.e., a person of skill in this art would understand that the use cycle must end with a detachment of the diaper so that the partially or fully used diaper can be disposed of). As an example, three (3) uses of the diaper would include: a first attachment of the diaper to the dog; a first detachment of the diaper from the dog; a second attachment of the diaper to the dog; a second detachment of the diaper from the dog; a third attachment of the diaper to the dog and a third detachment of the diaper from the dog. In this example, the diaper would be disposed of (as trash) after the third detachment from the dog.

The present invention includes, as a preferred embodiment, a diaper for dogs that is both reusable and disposable comprising:
a) a waterproof or water-resistant body sheet having a first end and a second end;
b) an absorbent material;
c) a fastener that attaches the first end of the body sheet to the second end of the body sheet;
   wherein the fastener can be attached, thereby attaching the first end of the body sheet to the second end of the body sheet, and detached, thereby releasing the first end of the body sheet from the second end of the body sheet, at least twice without damage to the fastener or the diaper so that the diaper can be used at least two times; and further wherein, in use, the diaper is fastened circumferentially about the rear torso of a dog and its inguinal region and does not encompass the base of the tail or cover the anus of the dog.

The present invention also includes, as a preferred embodiment, a diaper for dogs that is both reusable and disposable comprising:
a) a waterproof or water-resistant body sheet having a first end and a second end;
b) an absorbent material; and
c) a fastener that attaches the first end of the body sheet to the second end of the body sheet;
   wherein the fastener can be attached, thereby attaching the first end of the body sheet to the second end of the body sheet, and detached, thereby releasing the first end of the body sheet from the second end of the body sheet, at least twice without damage to the fastener or the diaper so that the diaper can be used at least two times; and further wherein, in use, the diaper is fastened circumferentially about the rear torso of a dog and its inguinal region and does not encompass the rear legs of the dog.

In use, the diaper of the present invention wraps around the rear torso of the dog so that all of the diaper is located between the front legs and rear legs of the dog (e.g., see FIG. 3).

As discussed earlier, the diaper of the present invention is designed so that the fastener can be attached and detached at least two times without damage to the fastener or the diaper so that the diaper can be used at least two times. In a preferred embodiment of the present invention, the diaper is designed so the fastener can be attached and detached at least three times without damage to the fastener or the diaper so that the diaper can be used at least three times. In other preferred embodiments of the present invention, the diaper is designed so that the fastener can be attached and detached at least four, five or six times without damage to the fastener or the diaper so that the diaper can be used at least four, five or six times, respectively.

Although many different types of fastener could be used in the diaper of the present invention, a preferred fastener is a hook and loop system (e.g., the fastener comprises cooperative Velcro-type fastening members).

In one embodiment of the present invention, the fastener comprises two parts and a first part of the fastener is attached to the first end of the body sheet and a second part of the fastener is attached to the second end of the body sheet and said fastener is completed by attaching the first and second parts of the fastener to each other.

The present invention also includes a method of using the diapers of the present invention, comprising the step of fastening the diaper circumferentially about the rear torso of a dog and its inguinal region so that all of the diaper is located between the front legs and rear legs of the dog and the diaper does not encompass the base of the tail or cover the anus of the dog.

Another method of using the diapers of the present invention that is encompassed by the present invention comprises the step of fastening the diaper circumferentially about the rear torso of a dog and its inguinal region so that all of the diaper is located between the front legs and rear legs of the dog and the diaper does not encompass the rear legs of the dog.

Another method of using the diapers of the present invention that is encompassed by the present invention comprises the steps of:
a) attaching the first end of the body sheet to the second end of the body sheet with the fastener so that: i) the diaper is fastened circumferentially about the rear torso of a dog and its inguinal region; ii) all of the diaper located between the front legs and rear legs of the dog; and iii) the diaper does not encompass the base of the tail or cover the anus of the dog;
b) detaching the fastener and removing the diaper from the dog before the absorbent material has been fully saturated with liquid;
c) reattaching the first end of the body sheet to the second end of the body sheet with the fastener so that: i) the diaper is fastened circumferentially about the rear torso of a dog and its inguinal region; ii) all of the diaper located between the front legs and rear legs of the dog; and iii) the diaper does not encompass the base of the tail or cover the anus of the dog;
d) detaching the fastener a second time and removing the diaper from the dog; and
e) disposing of the diaper.

The scope of the present invention should not be limited to the specific examples and descriptions provided in the foregoing specification. An artisan of ordinary skill will readily appreciate the numerous minor modifications that may be made to the present invention without departing from its spirit and scope.

What is claimed is:

1. A diaper for dogs that is both reusable and disposable comprising:
a) a waterproof or water-resistant body sheet having a first end and a second end;
b) an absorbent material;
c) a fastener that attaches the first end of the body sheet to the second end of the body sheet;
wherein said fastener can be attached, thereby attaching the first end of the body sheet to the second end of the body sheet, and detached, thereby releasing said first end of the body sheet from said second end of the body sheet, at least twice without damage to the fastener or the diaper so that the diaper can be used at least two times; and further wherein, in use, the diaper is fastened circumferentially about the rear torso of a dog and its inguinal region and does not encompass the base of the tail or cover the anus of the dog.

2. A diaper for dogs that is both reusable and disposable comprising:
a) a waterproof or water-resistant body sheet having a first end and a second end;
b) an absorbent material; and
c) a fastener that attaches the first end of the body sheet to the second end of the body sheet;
wherein said fastener can be attached, thereby attaching the first end of the body sheet to the second end of the body sheet, and detached, thereby releasing said first end of the body sheet from said second end of the body sheet, at least twice without damage to the fastener or the diaper so that the diaper can be used at least two times; and further wherein, in use, the diaper is fastened circumferentially about the rear torso of a dog and its inguinal region and does not encompass the rear legs of the dog.

3. The diaper of claim 2, wherein, in use, the diaper wraps around the rear torso of the dog so that all of the diaper is located between the front legs and rear legs of the dog.

4. The diaper of claim 1, wherein said fastener can be attached and detached at least three times without damage to the fastener or the diaper so that the diaper can be used at least three times.

5. The diaper of claim 2, wherein said fastener can be attached and detached at least three times without damage to the fastener or the diaper so that the diaper can be used at least three times.

6. The diaper of claim 1, wherein the fastener comprises a hook and loop system.

7. The diaper of claim 2, wherein the fastener comprises a hook and loop system.

8. The diaper of claim 1, wherein a first part of the fastener is attached to the first end of the body sheet and a second part of the fastener is attached to the second end of the body sheet and said fastener is completed by attaching the first and second parts of the fastener to each other.

9. A method of using the diaper of claim 1, comprising the step of fastening the diaper circumferentially about the rear torso of a dog and its inguinal region so that all of the diaper is located between the front legs and rear legs of the dog and the diaper does not encompass the base of the tail or cover the anus of the dog.

10. A method of using the diaper of claim 2, comprising the step of fastening the diaper circumferentially about the rear torso of a dog and its inguinal region so that all of the diaper is located between the front legs and rear legs of the dog and the diaper does not encompass the rear legs of the dog.

11. A method of using the diaper of claim 1, comprising the steps of:
a) attaching the first end of the body sheet to the second end of the body sheet with the fastener so that: i) the diaper is fastened circumferentially about the rear torso of a dog and its inguinal region; ii) all of the diaper located between the front legs and rear legs of the dog; and iii) the diaper does not encompass the base of the tail or cover the anus of the dog;
b) detaching the fastener and removing the diaper from the dog before said absorbent material has been fully saturated with liquid;
c) reattaching the first end of the body sheet to the second end of the body sheet with the fastener so that: i) the diaper is fastened circumferentially about the rear torso of a dog and its inguinal region; ii) all of the diaper located between the front legs and rear legs of the dog; and iii) the diaper does not encompass the base of the tail or cover the anus of the dog;
d) detaching the fastener a second time and removing the diaper from the dog; and
e) disposing of the diaper.

12. The diaper of claim 1, wherein said fastener can be attached and detached at least five times without damage to the fastener or the diaper so that the diaper can be used at least five times.

13. The diaper of claim 2, wherein said fastener can be attached and detached at least five times without damage to the fastener or the diaper so that the diaper can be used at least five times.

14. The diaper of claim 6, wherein the hook and loop system comprises cooperative Velcro-type fastening members.

15. The diaper of claim 7, wherein the hook and loop system comprises cooperative Velcro-type fastening members.
